(19) United States
(12) Patent Application Publication
(10) Pub. No.: US 2017/0216675 A1
(43) Pub. Date: Aug. 3, 2017

(54) FITNESS-BASED GAME MECHANICS

(71) Applicant: Disney Enterprises, Inc., Burbank, CA (US)

(72) Inventors: Michael P. GOSLIN, Sherman Oaks, CA (US); Joseph L. OLSON, Pasadena, CA (US)

(21) Appl. No.: 15/424,673

(22) Filed: Feb. 3, 2017

Related U.S. Application Data
(60) Provisional application No. 62/290,842, filed on Feb. 3, 2016.

(51) Int. Cl. A63B 24/00; A63F 13/213; A61B 5/11; G06T 19/00; G06F 3/01; A63F 13/211; A63B 71/06

(52) U.S. Cl. CPC A63B 24/0075; A63F 13/211; A63F 13/213; A63B 24/0006; A63B 71/0622; G06T 19/006; G06F 3/011; A61B 5/1118; A63B 2024/0068; A63B 2071/0638; A63B 2071/0636

(57) ABSTRACT

Embodiments provide techniques for rewarding users within a computer game for performing fitness activity. One embodiment determines a physical world fitness gaming objectives for a first user. Physical activity of the first user is monitored using one or more fitness devices to collect user fitness data. The user fitness data is analyzed to determine whether the first user has completed the one or more physical world gaming objectives. Upon determining a first one of the one or more physical world gaming objectives has been completed, embodiments determine one or more game rewards corresponding to the completed first physical world gaming objective and grant the first user the one or more game rewards within the first computer game.

### CROSS-REFERENCE TO RELATED APPLICATIONS

This application claims benefit of U.S. provisional patent application Ser. No. 62/290,842, filed Feb. 3, 2016, which is herein incorporated by reference in its entirety.

### BACKGROUND

Field of the Invention

The present invention generally relates to entertainment systems, and more specifically to techniques for providing fitness-based game mechanics within a computer gaming environment.

Description of the Related Art

Computer graphics technology has come a long way since video games were first developed. Relatively inexpensive 3D graphics engines now provide nearly photo-realistic interactive game play on hand-held video game, home video game and personal computer hardware platforms costing only a few hundred dollars. These video game systems typically include a hand-held controller, game controller, or, in the case of a hand-held video game platform, an integrated controller. A user interacts with the controller to send commands or other instructions to the video game system to control a video game or other simulation. For example, the controller may include a joystick and buttons operated by the user.

While video games allow the user to interact directly with the video game system, such interactions primarily influence the graphical depiction shown on the video game device (or on a connected display), and rarely influence any other objects outside of the virtual world. That is, a user may specify an input to the video game system, indicating that the user's avatar should perform a jump action, and in response the video game system could display the user's avatar jumping. However, such interactions are typically limited to the virtual world, and any interactions outside the virtual world are limited (e.g., a hand-held gaming device could vibrate when certain actions occur).

Modern technologies such as augmented reality devices enable game developers to create games that exist outside of traditional video game platforms (e.g., where the virtual world is solely output through a display device). Using such technologies, virtual characters and other virtual objects can be made to appear as if they are present within the physical world. In such augmented reality experiences, it is generally preferable for the virtual character to be rendered with realistic dimensions and positioning, in order to enhance the illusion that the characters are truly present within the physical world.

### SUMMARY

Embodiments provide a method, non-transitory computer-readable medium and system for rewarding users within a computer game, for physical activity performed outside the computer game. The method, non-transitory computer-readable medium and system include determining one or more physical world fitness gaming objectives for a first user in a first computer game. The method, non-transitory computer-readable medium and system also include monitoring physical activity of the first user using one or more fitness devices to collect user fitness data. Additionally, the method, non-transitory computer-readable medium and system include analyzing the user fitness data collected from the one or more fitness devices to determine whether the first user has completed the one or more physical world gaming objectives. The method, non-transitory computer-readable medium and system further include, upon determining a first one of the one or more physical world gaming objectives has been completed, determining one or more game rewards corresponding to the completed first physical world gaming objective, and granting the first user the one or more game rewards within the first computer game.

### BRIEF DESCRIPTION OF THE DRAWINGS

So that the manner in which the above recited aspects are attained and can be understood in detail, a more particular description of embodiments of the invention, briefly summarized above, may be had by reference to the appended drawings.

It is to be noted, however, that the appended drawings illustrate only typical embodiments of this invention and are therefore not to be considered limiting of its scope, for the invention may admit to other equally effective embodiments.

FIG. 1 illustrates an environment in which a user interacts with a game system using electronic devices, according to one embodiment described herein.

FIG. 2 is a flow diagram illustrating the incorporation of physical world fitness gaming objectives into a computer game, according to one embodiment described herein.

FIG. 3 illustrates a physical environment including storytelling devices and a user, according to one embodiment described herein.

FIG. 4 is a block diagram illustrating a fitness device, according to one embodiment described herein.

FIG. 5 is a flow diagram illustrating a method of granting game reward to a user based on physical activity, according to one embodiment described herein.

FIG. 6 is a flow diagram illustrating a method of rewarding users for physical activity, according to one embodiment described herein.

FIG. 7 is a block diagram illustrating an interactive object, according to one embodiment described herein.

FIG. 8 is a block diagram illustrating a controller device, according to one embodiment described herein.

FIG. 9 is a block diagram illustrating a mobile device configured with an augmented reality component, according to one embodiment described herein.

FIG. 10 is a block diagram illustrating an augmented reality headset, according to one embodiment described herein.

### DETAILED DESCRIPTION

Embodiments described herein generally provide game mechanics based on fitness metrics collected from a user-carried fitness device. For example, such a fitness device could be a wristband that is worn by the user and that includes sensor devices capable of tracking the user's behavior. Such sensor devices could include, for example, accelerometers, inertial measurement unit (IMU) sensors, electromyography (EMG) sensors, heart rate sensors, and so on. A fitness game component (e.g., software executing on one or more computing devices) could receive fitness metrics collected from the user-carried fitness device and could alter one or more gameplay elements based on the fitness metrics. For instance, a particular game could have the user perform one or more training activities as part of an in-game quest. Upon receiving fitness data from the fitness device indicating that the user has performed a sufficient level of physical activity to satisfy the quest's objective, the fitness game component could complete the quest within the game and could provide the user with the associated reward.

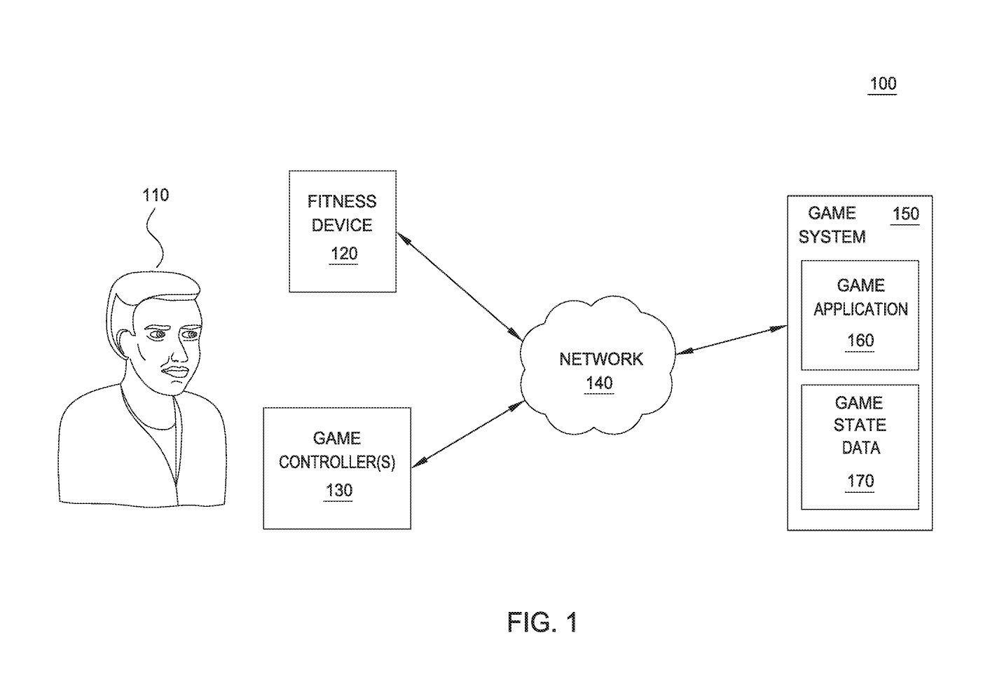

FIG. 1 illustrates an environment in which a user interacts with a game system using electronic devices, according to one embodiment described herein. As shown, the environment 100 includes a user 110, a fitness device 120, game controller(s) 130, and a game system 150, interconnected via a network 140. The game system 150 includes a game application 160 and game state data 170. Generally, the game application 160 represents a software application for a computer game with one or more physical world fitness objectives. The game state data 170 generally represents data maintained by the game application 160 for users playing the computer game. For example, the game state data 170 could specify information describing a user avatar (e.g., the avatar's appearance, obtained items, level, special abilities, attributes, etc.) within the computer game. Generally, the game controller(s) 130) represents an input device through which the user can provide inputs for controlling the game application 160.

The fitness device 120 represents a device capable of monitoring physical activity of the user 110. For example, the fitness device 120 could include one or more sensor devices such as accelerometers, IMU sensors, EMG sensors, heart rate sensors, and so on. In one embodiment, the fitness device 120 is configured to be worn by the user 110. For example, the fitness device 120 could be fitted with a clasp that the user can attach to, e.g., an article of clothing. In one embodiment, the fitness device 120 is configured to be worn by the user 110 (e.g., on the user's wrist as a bracelet or watch). More generally, the fitness device 120 represents any device with sensors (or capable of communicating with sensors) capable of monitoring fitness metrics of a user.

According to one embodiment, the game application 160 is configured to determine one or more physical world fitness gaming objectives for the user 110. For example, the game application 160 could determine that the one or more physical world fitness gaming objectives include walking (or otherwise travelling) a number of steps or distance, performing a number of physical exercises (e.g., push-ups, sit-ups, jumping jacks, etc.) and so on. The game application 160 could then monitor physical activity of the first user using one or more fitness devices to collect user fitness data. For example, the game application 160 could configure the fitness device 120 to monitor the user's 110 physical activity, using sensor devices of the fitness device 120. By configuring the fitness device 120 in this way, the user's activity can be monitored, even when the user 110 is away from the game system 150. That is, while the game system 150 may not be particularly portable (e.g., a console gaming system connected to a display device), the game application 160 can configure the fitness device 120 (which may be highly portable) to monitor the user's activity and to collect fitness data describing the user's activity, even while the user 110 is away from the game system 150. For example, the fitness device 120 could collect fitness data describing the user's activity while the user is out of the house, even though the game system 150 may remain stationary within the user's house. The game application 160 could then retrieve the fitness data from the fitness device 120, when the user is again proximate to the game system 150.

The game application 160 could then analyze data collected from the one or more fitness devices to determine whether the first user has completed the one or more physical world gaming objectives. For example, a particular mission for the game application 160 could task the user 110 with performing 50 push-ups and walking 10,000 steps (i.e., within the real world), in order to unlock one or more game rewards (i.e., within the virtual world). Upon determining a first one of the one or more physical world gaming objectives have been completed, the game application 160 could determine one or more game rewards corresponding to the completed first physical world gaming objective and the game application 160 could grant the first user the one or more game rewards within the first computer game. As an example, upon determining that user has completed the tasked physical world gaming objectives, the game application 160 could increase one or more physical attributes of the user's avatar within the computer game.

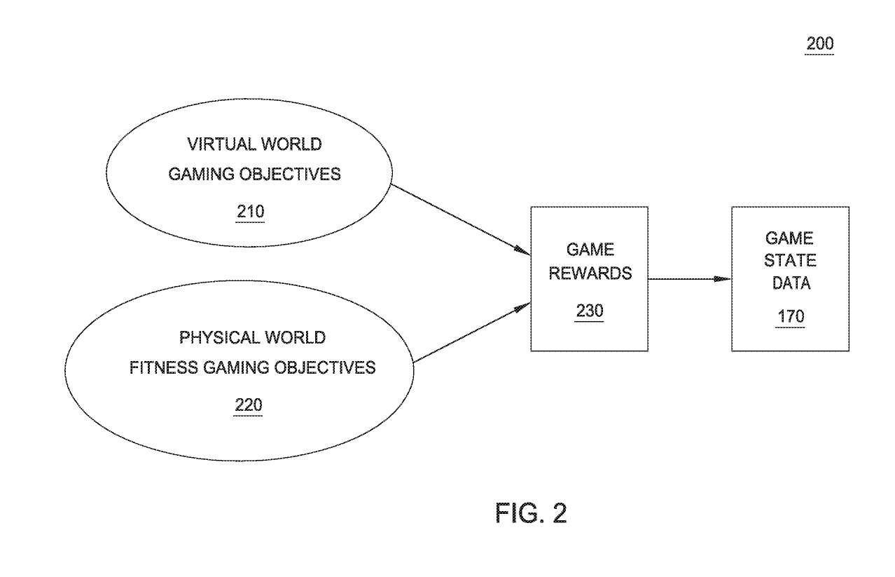

FIG. 2 is a flow diagram illustrating the incorporation of physical world fitness gaming objectives into a computer game, according to one embodiment described herein. As shown, the flow diagram includes virtual world gaming objectives 210, physical world fitness gaming objectives 220, game rewards 230 and game state data 170. As discussed above, the game state data 170 can include information for a user's avatar within a virtual world. Such information can include, for instance, attributes of the avatar, abilities of the avatar, an appearance of the avatar, and so on. The virtual world gaming objectives 210 represent tasks, quests, missions and the like that the user can complete within the virtual world. As shown, upon completion of a virtual world gaming objective 210, the game application 160 can determine a corresponding game reward from the game rewards 230 and can update the game state data 170 to grant the determined game reward to the user. For example, upon the completion of a particular mission virtual world gaming objective 210, the game application 160 can determine a number of experience points having a predefined relationship with the particular mission and can grant the experience points to the user's avatar.

Additionally, the game application 160 can track the user's progress in completing the physical world fitness gaming objectives 220. For example, one such physical world fitness gaming objective could be a mission to walk 10,000 steps, while another physical world fitness gaming objective could be to travel to a gym and to perform a workout where the user's heart exceeds 140 beats per minute. The game application 160 could then monitor the user's progress in completing the physical world fitness gaming objectives 220 using one or more fitness devices 120. For instance, one of the fitness devices could include an IMU, accelerometer, and/or other sensor and logic to analyze the collected sensor data and to determine when the data is representative of the user walking a step. The logic within the fitness device could maintain a count of how many steps the user has walked (e.g., since the user was tasked with completing the physical world fitness gaming objective 220).

Upon determining that the user has walked a threshold number of steps having a predefined association with the physical world fitness gaming objective 220, the game application 160 could determine that the physical world fitness gaming objective 220 has been completed. The game application 160 could then determine one or more game rewards 230 that correspond to the physical world fitness gaming objective 220, and the game application 160 could update the game state data 170 to reflect that the determined game rewards 230 have been granted to the user's avatar. For example, upon determining that the user has completed the fitness gaming objective of walking 10,000 steps, the game application 160 could determine that the fitness gaming objective corresponds to a game reward 230 of increased endurance for the user's avatar, and could update the game state data 170 to assign the increased endurance ability to the user's avatar. Such a correspondence between physical world fitness gaming objectives 220 and game rewards 230 could be specified, e.g., within a database accessible by the game application 160.

As another example, the game application 160 could assign the user with the physical world fitness gaming objective 220 of travelling to a gym and getting the user's heartbeat over 140 beats per minute (BPM). In doing so, the physical world fitness gaming objective 220 could configure a first fitness device 120 (e.g., a mobile device carried by the user) to monitor the user's real world position (e.g., using one or more Global Positioning System (GPS) transceivers). The game application 160 could compare the monitored position of the user with map data describing physical locations and the game application 160 could analyze metadata corresponding to the physical locations to determine when the user has completed the objective of travelling to a gym. For example, the game application 160 could determine a user's location expressed as a set of coordinates, and the game application 160 could determine a physical location corresponding to the set of coordinates using predefined map data. The game application 160 could then access metadata describing the physical location to determine whether the physical location corresponds to a gymnasium.

Additionally, the game application 160 could configure another fitness device 120 (e.g., a heartrate monitor) to track the user's heartbeat and to collect fitness data describing the user's progress in completing the assigned physical world fitness gaming objective. As an example, upon monitoring the user's heartbeat and determining the user's heartbeat has exceeded 140 BPM, logic on the fitness device could log the time and duration that the user's heartbeat exceeded 140 BPM. This logged data could subsequently be retrieved by the game application 160 and cross-referenced with the positional data collected by the first fitness device. For instance, if the game application 160 determines that the user was at a physical location classified as a gymnasium (e.g., based on the analysis of metadata corresponding to the user's location) for a particular window of time, the game application 160 could analyze the collected heartrate data and could determine whether the user's heartbeat exceeded 140 BPM during the window of time. For example, if the game application 160 determines that the user's heartbeat exceeded 140 BPM while the user was at the physical location determined to be a gymnasium, the game application 160 could determine that the user has completed the physical world fitness gaming objective 220 and could determine a game reward 230 corresponding to the physical world fitness gaming objective 220. For example, the game application 160 could determine that the fitness gaming objective corresponds to an increase in the strength attribute of the user's avatar, and could update the game state data 170 for the user's avatar to increase the avatar's strength attribute accordingly.

Particular embodiments are described herein with respect to an immersive storytelling environment in which a story is played back through the interaction of storytelling devices (also referred to as interactive devices). More specifically, embodiments may use various storytelling devices, each capable of producing some auditory and/or visual effect, to create an immersive and interactive storytelling experience for a user. Such a system may include a variety of storytelling devices and a controller, connected via a network (e.g., an RF communications network). Each storytelling device generally represents any device capable of enhancing a storytelling experience, in response to user input (or some stimuli) a current context of a story. For instance, the game application 160 could act as a controller component for such a storytelling environment, and the game application 160 could configure the storytelling devices with stimulus and response information, based on a current context of a story. As an example, the game application 160 could configure a particular storytelling device to generate audiovisual messages responsive to a certain stimulus event (e.g., a user performing a particular action), and to perform another action responsive to other stimulus (e.g., the user not performing the particular action within a predefined window of time).

Additionally, embodiments can include augmented reality devices together with various storytelling devices as part of an augmented reality gaming environment. As used herein, an augmented reality device refers to any device capable of displaying a real-time view of a physical, real-world environment while altering elements within the displayed view of the environment. As such, unlike a virtual reality device which displays a view of virtual world, an augmented reality device displays a view of the real world but augments elements using computer graphics technology. For example, the game application 160 could execute on or in coordination with an augmented reality device may include a camera device (or multiple camera devices) used to capture a view of the real-world environment and may further include computer software and/or hardware configured to augment elements of the captured scene. For example, the game application 160 could capture a series of images of a coffee cup sitting on top of a table, modify the series of images so that the coffee cup appears as an animated cartoon character and display the modified series of images in real-time to a user. As such, when the user looks through the augmented reality device, the user sees an augmented view of the physical real-world environment in which the user is located.

Additionally, the game application 160 could identify a first physical object within the visual scene captured by camera devices of the augmented reality device. For instance, the game application 160 could analyze the visual scene to determine the border edges of objects within the visual scene, and could use these border edges in order to identify one or more physical objects existing within the visual scene. Of note, as the captured visual scene represents a three-dimensional space (e.g., a physical environment captured using a camera of the augmented reality device), the game application 160 may be configured to estimate a three-dimensional space occupied by each of the physical objects within the captured scene. That is, the game application 160 could be configured to estimate the three-dimensional surfaces of physical objects within the captured scene.

In response to detecting a known physical object with the visual scene, the game application 160 could render one or more virtual characters based on the physical object's appearance within the captured frames. As an example, the game application 160 could create a three-dimensional representation of the physical environment and could create a virtual object or character to insert within the three-dimensional representation. The game application 160 could position the created virtual object or character at a position within the three-dimensional scene, based on the depiction of the physical object within the captured frames. For example, the game application 160 could determine that the physical object is resting on a particular surface within the physical environment (e.g., a table surface, a floor, etc.), based on data about the size and shape of the physical object and the object's appearance within the captured frames. Upon identifying the physical surface, the game application 160 could position the virtual object or character within the three-dimensional scene, so that the virtual object or character is resting on the identified surface.

In doing so, the game application 160 could scale the size of the virtual object or character based on the depiction of the physical object within the captured frames. For instance, the game application 160 could store predefined geometric data for the physical object, specifying a shape and dimensions of the physical object. The game application 160 could then use such information to determine how to size the virtual object or character in the three-dimensional scene. For example, assume the virtual object is a spherical object that is 12 inches in diameter. The game application 160 could determine a scaling for the virtual object based on the size of the physical object within the captured frames and the predefined geometric data specifying the physical object's known dimensions. As another example, the game application 160 could create a virtual character and could scale the size of the virtual character to life-size dimensions (e.g., the size of an average human being), using the size of the physical object within the captured frames and the predefined geometric data specifying the physical object's known dimensions. Doing so enables the game application 160 to create a realistic and consistent depiction of the virtual object or character.

Generally, the game application 160 can continue rendering frames of the three-dimensional scene interlaced with the frames captured by the camera sensors of the augmented reality device, in real-time, as the device (and the user of the device) moves throughout the physical environment. Advantageously, doing so provides a more immersive augmented reality experience for the user, as the user can paint the surfaces of objects within the augmented reality world and the user's painting will persist and remain accurate to the depicted physical environment, even when the environment is viewed from different perspectives using the augmented reality device.

As an example, the game application 160 could render frames depicting a virtual character within the physical environment, and could depict the virtual character assigning a physical world fitness gaming objective 220 to the user. The game application 160 could simultaneously output audio data with dialogue for the virtual character. Upon receiving user input accepting the physical world fitness gaming objective 220, the game application 160 could configure one or more fitness devices 120 to monitor one or more fitness metrics corresponding to the physical world fitness gaming objective 220 using one or more sensor devices. For example, the game application 160 could configure a fitness device to monitor sensor data collected from one or more sensor devices and to determine a number of fitness events (e.g., steps) the user has performed, based on portions of the sensor data matching a predefined pattern of sensor data.

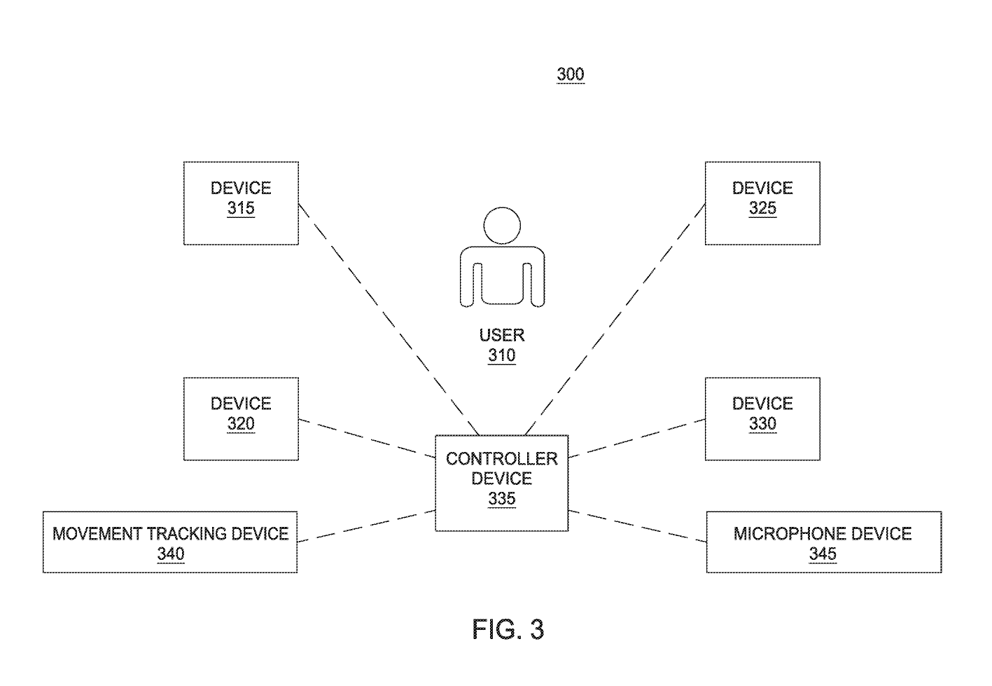

FIG. 3 illustrates a physical environment including storytelling devices and a user, according to one embodiment described herein. As shown, the environment 300 includes a user 310 surrounded by a number of storytelling devices 315, 320, 325 and 335 as well as a control device 335. The environment 300 further includes a movement tracking device 340 and a microphone device 345. For example, the movement tracking device 340 could represent one or more camera devices, an electromyography sensor device, and so on, and more generally represents any electronic device capable of collecting data through which a user's movement can be determined. The storytelling devices 315, 320, 325 and 335 may represent fictional characters, e.g., super heroes within a particular fictional storyline. Generally, the control device 335 can control the behavior (e.g., the movement, audio output, etc.) of the devices 315, 320, 325 and 330 as part of a computer gaming experience. For example, the control device 335 can control the behavior of the devices 315, 320, 325 and 330 in assigning a physical world fitness gaming objective 220 to the user, such that audio effects representing dialogue from characters the devices 315, 320, 325 and 330 represent are output using one or more speaker devices.

In one embodiment, the control device 335 is configured to select two or more of the devices 315, 320, 325 and 330 to output a particular sound and can generate a schedule by which the selected devices should output the sound. For instance, such a schedule could specify that the selected devices should output the sound in unison or could specify that each of the selected devices should output sound effects at different points in time. In one embodiment, the devices are configured to output the same sound effect at different points in time, so as to introduce a time delay between the audio output of each device. For example, a particular story having a jungle theme could include ambient sound effects that simulate the sounds of a jungle, including birds chirping, insects buzzing, the sound of a distant waterfall, and so on. In outputting the ambient sound effects, the control device 335 could distribute the various sound effects across the devices 315, 320, 325 and 330 (with some potentially output by the control device 335 itself) and could generate a timing schedule by which the various sound effects should be played by the devices 315, 320, 325 and 330. For example, the schedule could specify that the sound effects should be temporally staggered (i.e., not all played at the same time) and could distribute the sound effects across the devices 315, 320, 325 and 330, so as to create a three-dimensional soundscape for the user 310.

In one embodiment, the control device 335 is configured to consider the position of the user 310 relative to the position of the devices 315, 320, 325 and 330, when distributing and scheduling sound effects to the various devices 315, 320, 325 and 330. For instance, assume that a particular story takes place within a bee hive and includes ambient sound effects simulating bees flying all around the user 310. The controller 335 could consider the user's 310 position in distributing the ambient sound effects to the devices 315, 320, 325 and 330 for playback, so as to ensure the output of the sound effects creates an immersive and three-dimensional soundscape for the user. Thus, in this example, the controller 335 could schedule the sound of a bee buzzing to be output by each of the devices 315, 320, 325 and 330 with a time delay in between each output, so that the sound of the bee appears to repeatedly encircle the user 310 who is positioned roughly in between all of the devices 315, 320, 325 and 330.

Moreover, the controller 335 can be configured to dynamically update the playback schedule and the devices used in the playback in real-time, as the position of the user 310 and the various devices changes. For instance, as the devices move throughout the physical environment (e.g., when carried by a user, when moving on their own, etc.), the controller 335 could dynamically update the playback schedule of the bee buzzing sound effect to maintain the effect of the sound encircling the user 310. For example, a first sequential playback order for the bee buzzing sound effect could be devices 315, device 320, control device 335, device 330 and then device 325, which could repeat indefinitely provided the devices 315, 320, 325, 330 and 325 and the user 310 remain in their depicted positions. However, if as part of the story playback the devices 315 and 330 move throughout the physical environment and change positions, the control device 335 could update the sequential playback order to be device 330, device 320, control device 335, device 315 and then device 325.

In one embodiment, a game application 160 is configured to determine one or more physical world fitness gaming objectives for a first user 310 in a first computer game. In the environment 300, the controller device 335 may represent logic within the game application 160 executing on a game system 150 that is configured to transmit instructions to and otherwise control the behavior of the devices 315, 320, 325 and 330. The physical world fitness gaming objective could be determined, for example, based on a current content of the computer game. For instance, the game application 160 could be configured to provide a particular virtual location that offers particular objectives to user avatars in the virtual location. As such, the user 310 could travel to the virtual location with the user's avatar and could be assigned the physical world fitness gaming objective.

As another example, the game application 160 could make a number of physical world fitness gaming objectives available to users through a user interface. For instance, such a graphical interface could specify the fitness activity needed to complete the objective (e.g., walk a certain distance, walk a certain number of steps, achieve a heartrate above a certain BPM, perform an exercise a number of times, etc.). Additionally, the interface could indicate a game reward(s) that can be earned by completing each of the fitness objectives. In such an example, the user could select a physical world fitness gaming objective that the user wishes to complete using an input device of the game application 160 (e.g., a game controller 130, movement tracking device 340, microphone device 345, etc.).

The game application 160 then monitors physical activity of the first user using one or more fitness devices to collect user fitness data. For example, the game application 160 could assign the user a physical world fitness gaming object of performing 25 jumping jack exercises in order to gain an in-game virtual reward (e.g., an increase in the endurance attribute of the user's avatar, a defined number of experience points, etc.) and the game application 160 could monitor the user's physical activity using a movement tracking device 340. The game application 160 could then analyze data collected by the movement tracking device 340 to determine when the user completes the activity. For instance, where the movement tracking device 340 is a camera device, the game application 160 could analyze frames of captured video data to identify the user within the frames and to determine the user's movement across an interval of time. As another example, where the movement tracking device 340 is an EMG sensor device, the game application 160 could analyze electromyograms collected by the EMG sensor device to determine when the electromyograms match a predefined pattern of EMG sensor data that corresponds to a jumping jack exercise.

Upon determining that a first one of the one or more physical world gaming objectives has been completed, the game application 160 could determine one or more game rewards corresponding to the completed first physical world gaming objective and could granting the first user the one or more game rewards within the first computer game. For example, upon analyzing the sensor data and determining that the user has performed 25 jumping jacks, the game application 160 could update game state data 170 for the user's avatar to grant the user the corresponding reward.

In one embodiment, the game application 160 is configured to apply a game penalty to the first user within the first computer game, upon determining that the user has failed to complete the one or more physical world gaming objects within a defined window of time. For instance, the game application 160 could assign the user the physical world gaming objective of walking 10,000 steps per day, and upon determining that the user has not completed the assigned task within the designated period of time, the game application 160 could impose a penalty on the user's avatar within the computing game. As an example, the game application 160 could penalize the user by “damaging” the user's avatar within the computer game by a determined amount of health. In one embodiment, the game application 160 is configured to determine the amount of health (or life points) to deduct from the user's avatar, based on the collected fitness data for the user during the designated time period. For instance, if the game application 160 determines that the user walked 9,500 of the 10,000 assigned steps during the designated window of time, the game application 160 could deduct a relatively small amount of health from the user's avatar, as the user was very close to completing the assigned fitness objective. On the other hand, if the game application 160 determines that the user only walked 1,500 of the 10,000 assigned steps during the designated window of time, the game application 160 could deduct a relatively larger amount of health from the user's avatar, as the user was well under the assigned fitness goal. In other words, in one embodiment, the game application 160 can scale the amount of damage inflicted to the user's avatar by an amount that is proportional to the difference between the assigned fitness goal and the measured fitness activity for the user.

Of note, while the above example involves deducting health from the user's avatar's health pool, more generally the game application 160 can adversely affect any number of user avatar attributes (or more generally, gaming attributes) relating to the user. For example, the game application 160 could be configured to alter an appearance of the user, in response to the user failing to complete the fitness objective (e.g., by depicting the avatar as gaining weight). As another example, the game application 160 adversely affect the avatar's performance within the game, based on the user's failure to complete the physical world fitness objective. For example, in a sports computer game, the game application 160 could reduce the user's avatar's physical attributes (e.g., strength, speed, acceleration, passing, blocking, etc.) by a determined amount (e.g., a predefined amount, an amount determined based on the difference between the user's performance and the assigned fitness objective, etc.). In one embodiment, the game application 160 can adversely affect one or more virtual objects associated with the user. For example, the health of the user's virtual pet could be reduced, based on the user's failure to accomplish the fitness objective within the specified window of time.

In a particular embodiment, the game application 160 is configured to provide additional game rewards to the user for exceeding the assigned fitness objective. For instance, if the user is assigned to talk 10,000 steps and instead walks 25,000 steps during the assigned time period, the game application 160 could increase the game reward associated with the assigned fitness objective. As an example, the game application 160 could scale the game reward by an amount by which the user exceeded the assigned fitness objective (e.g., a +150% bonus could be applied to the game reward, as the user exceeded the assigned number of steps by 15,000 steps). For example, the game application 160 could grant an amount of health to the user's avatar (or the user's virtual pet), based on the amount of additional health associated with completing the fitness object and a multiplier based on the amount the user's tracked fitness data exceeded the assigned goal.

In one embodiment, the game application 160 is configured to provide one or more stretch goals for the assigned fitness objective that, if completed by the user (e.g., within a designated window of time), result in additional game rewards. For example, the game application 160 could assign the user the fitness objective of walking 10,000 steps in a day, which corresponds to a game reward of additional health for the user's avatar. Additionally, the game application 160 could provide a stretch goal of 15,000 steps in the day, which corresponds to a game reward of additional strength (e.g., permanently, for a fixed duration, etc.), and a second stretch goal of 20,000 steps in the day, which corresponds to a game reward of additional health and strength for the user's virtual pet within the game world. As such, if the game application 160 determines that the user managed to walk at least 20,000 steps in the day, the game application 160 could grant the user not only the additional health for completing the assigned objective, but could additionally grant the user the additional strength and could grant additional health and strength to the user's virtual pet within the game.

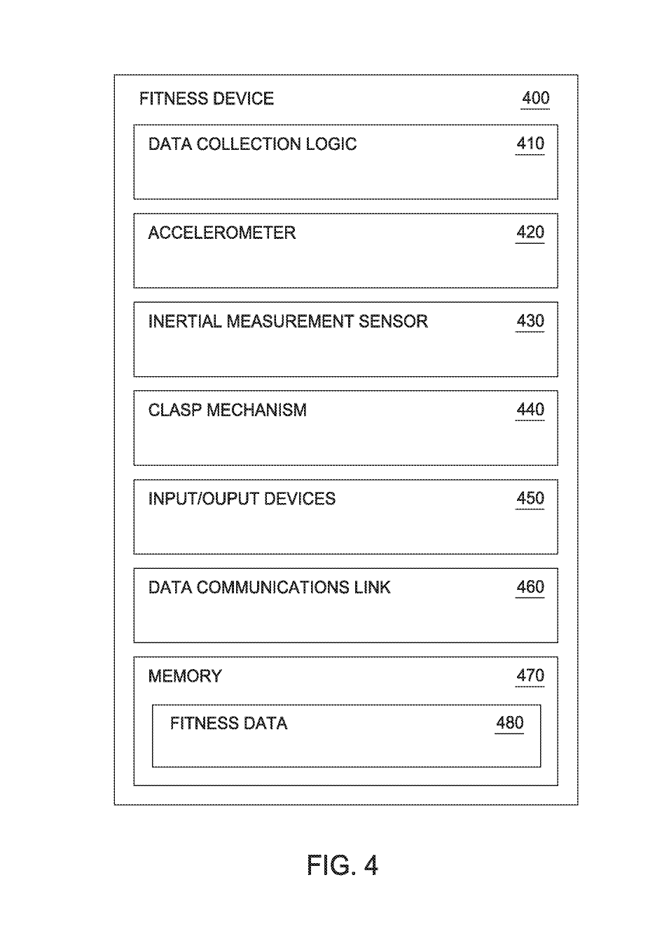

FIG. 4 is a block diagram illustrating a fitness device, according to one embodiment described herein. As shown, the fitness device 400 includes data collection logic 410, an accelerometer device 420, an inertial measurement sensor (IMU) 430, a clasp mechanism 440, one or more input and/or output devices 450, a data communications link 460 and a memory 470. Generally, the data collection logic 410 represents computerized logic configured to collect data from the accelerometer device 420, the IMO 430 and the input and/or output devices 450. The memory contains fitness data 480, representing data gathered by the data collection logic 410. For example, the data collection logic 410 could be configured to monitor data collected by the IMU 430 to determine when the collected data matches a predefined pattern of data representing a step taken by the user. Continuing the example, the fitness data 480 could contain a count of approximated steps taken by the user, and upon determining that collected data matches the predefined pattern, the data collection logic 410 could increment the count within the fitness data 480.

The controller device 335 could then retrieve the fitness data 480 from the memory 470 (e.g., over the data communications link) and the controller device 335 could use the collected data as part of a gameplay mechanic. For example, the controller device 335 could manage an augmented reality game in which the user is tasked with performing in-game training and the controller device 335 could instruct the user (e.g., by controlling one or more physical storytelling devices, by controlling one or more virtual characters depicted by an augmented reality device, etc.) to perform a particular set of physical activities as part of the in-game training. In such an example, the controller device 335 could track the user's behavior using the sensor devices within the fitness device 400 and, upon determining the user has performed the particular set of physical activities, the controller device 335 could reward the user with a corresponding reward. For instance, the controller device 335 could grant the user a predefined number of experience points for successfully completing an assigned number of physical tasks (e.g., push-ups, jumping jacks, steps, etc.). Such experience points could enable the user to unlock certain abilities, skills and powers within the gaming experience.

As another example, the controller device 335 could assign the user a task of performing a particular physical gesture (e.g., a hand gesture) corresponding to a particular in-game ability (e.g., a telepathic ability). For example, the user could unlock the ability to perform the particular in-game ability by completing one or more assigned physical activities while wearing the fitness device 400. The controller device 335 could then monitor data collected sensors (e.g., IMU 430, accelerometer 420, an electromyography sensor, etc.) within the fitness device 400 to detect when the user has performed the physical gesture. Upon detecting the user has performed the gesture, the controller device 335 could perform a corresponding in-game effect. For instance, the controller device 335 could render a plurality of frames, depicting a virtual effect corresponding to the performed gesture and could output such frames for display on an augmented reality device. For instance, if the performed gesture corresponds to a telepathic mind trick, the controller device 335 could render frames depicting a virtual character being placated in response to the user's telepathic ability. Doing so creates a more immersive experience for the user, as the user's physical actions are used to control the virtual game world.

Additionally, the controller device 335 can use the fitness device 400 to track a user's lack of action, as part of a gameplay experience. For example, the controller device 335 could task the user with performing a meditation activity for a defined period of time (e.g., 1 minute). During this time, the controller device 335 could monitor data collected by the fitness device 400 describing the user's physical movements to determine whether the user is holding sufficiently still to complete the assigned task. Upon determining that the user has been sufficiently still, the controller device 335 could notify the user that the user has successfully completed the assigned gameplay task (e.g., by rendering one or more frames depicting a virtual character congratulating the user and by outputting accompanying audio) and could award the user within the gaming environment (e.g., awarding the user experience points for successfully completing the task).

In one embodiment, the controller device 335 is configured to control the devices within the gaming environment in a predefined manner during the user's assigned task. For example, a particular task could require the user to stay in bed for a set period of time, while spooky music and images are shown within the physical environment. As such, the controller device 335 could be configured to output a musical audio track(s) using an output device within the physical environment and could be configured to create spooky augmented reality effects within the physical environment.

Additionally, the fitness device 400 could store state information describing the user's current status within the gaming environment. Such state information could include a level of the user, abilities unlocked by the user, a faction the user has joined, and so on. The fitness device 400 could provide an Application Program Interface (API) that allows external devices to access this state information. As an example, an external device at a theme park could access a user's state information from a user-worn fitness device and could then use such information to track the user's behavior outside of the gaming environment.

For instance, an external device could access a user's state information and could determine that the state information indicates the user has allied with a particular faction's forces within the gaming experience. The external device could then unlock additional experiences for the user (e.g., within an attraction at the theme park), based on the user's state information. For instance, upon determining that the state information indicates that the user has allied with a particular fictional faction's forces within the gaming environment, the external device could output an audio effect at the theme park, greeting the user in a faction-appropriate manner when the user approaches themed attraction corresponding to the faction. Other examples include the external device instructing the fitness device 400 to provide haptic feedback, indicating that special content is available to the user as a result of the state information. By enabling the user to interact with the theme park environment based on the user's gaming state information and in ways that weren't possible previously, embodiments provide a more immersive experience for the user.

Additionally, upon determining that a user has visited a particular attraction at the theme park, the external device could update the state information on the fitness device 400 (e.g., using the API). The state information could then be used (e.g., by controller device 335) to alter the gameplay experience. For instance, upon determining that the state information indicates that a user has visited a particular attraction within a theme park, the controller device 335 could provide a reward to the user within the gaming environment. As an example, the controller device 335 could unlock an in-game ability for the user based on the user visiting the theme park. As another example, the controller device 335 could award the user with a predefined number of experience points.

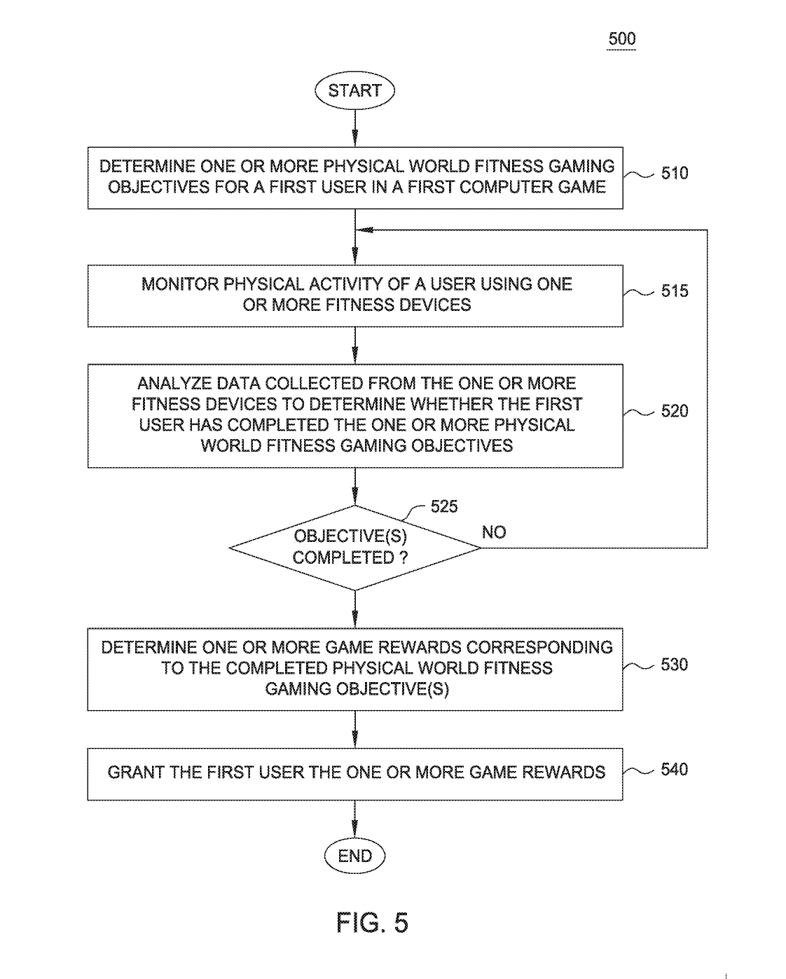

FIG. 5 is a flow diagram illustrating a method of granting game reward to a user based on physical activity, according to one embodiment described herein. As shown, the method begins at block 500, where a game application 160 determines one or more physical world fitness gaming objectives for a first user in a first computer game. The game application 160 then monitors physical activity of the first user using one or more fitness devices (block 515). Additionally, the game application 160 analyzes data collected from the one or more fitness devices to determine whether the first user has completed the one or more physical world fitness gaming objectives (block 520). For example, the game application 160 could configure a particular fitness device to track how many steps the first user walks. In doing so, the game application 160 could configure the fitness device to analyze IMU and/or accelerometer sensor data to detect when the sensor data matches a predefined pattern of sensor data indicative of a user walking a step while carrying the fitness device on the user's person. As another example, the game application 160 could analyze EMG data to detect when a portion of the EMG data matches a predefined pattern indicative of performing a particular physical activity (e.g., a push-up exercise).

At some subsequent point in time, the game application 160 determines whether the physical world fitness gaming objectives have been completed (block 525). If the objectives are not yet completed, the method returns to block 515, where the game application 160 continues monitoring the physical activity of the user using the one or more fitness devices. On the other hand, if the game application 160 determines that at least one of the physical world fitness gaming objectives has been completed, the game application 160 determines one or more game rewards corresponding to the gaming objective (block 530). For instance, the game application 160 could access a mapping data structure that maps gaming objectives to game rewards. As an example, the game application 160 could query the mapping data structure using an identifier that uniquely identifies the completed physical world fitness gaming objective within the game application 160 to determine the one or more corresponding game rewards. The game application 160 grants the determined game rewards to the first user (block 540), and the method 500 ends.

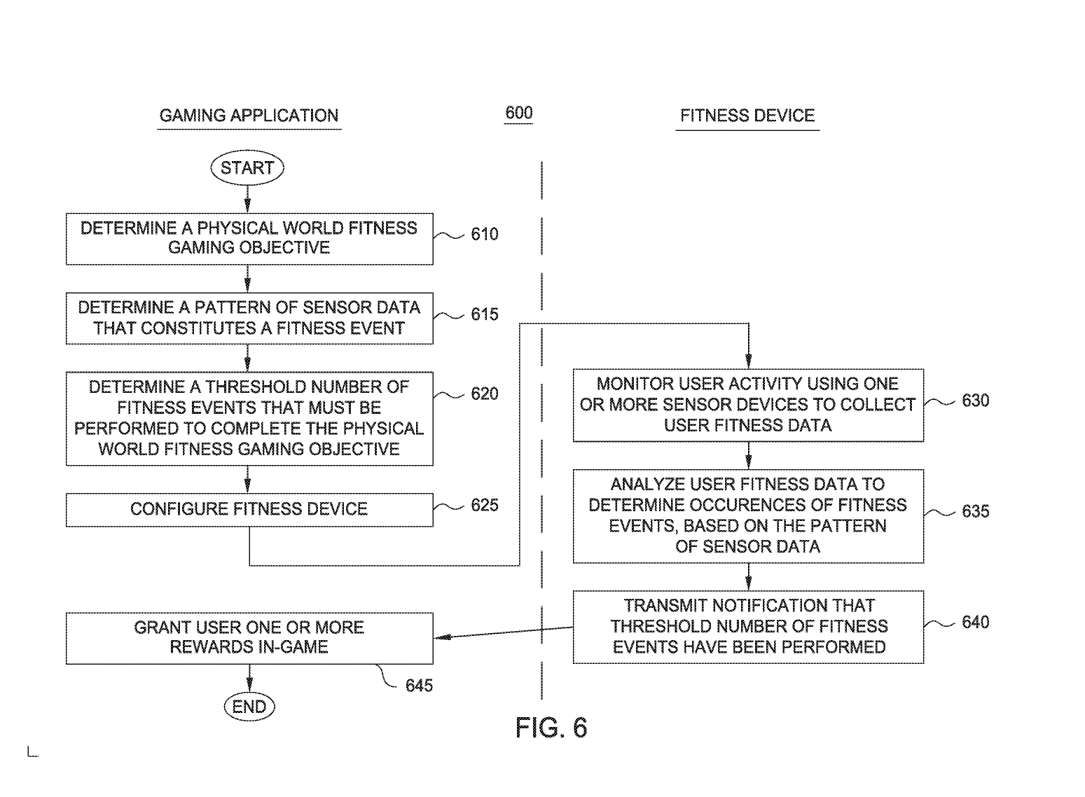

FIG. 6 is a flow diagram illustrating a method of rewarding users for physical activity, according to one embodiment described herein. As shown, the method 600 begins at block 610, where the game application 160 determines a physical world fitness gaming objective. Examples of such objectives include, without limitation, walking a defined number of steps, performing a particular exercise activity a defined number of times, achieving a heartbeat above a particular rate, remaining sufficiently inactive for a defined period of time (e.g., a meditation activity), and so on. Additionally, such an objective may specify conditions under which the physical activity must be performed. As an example, the objective may specify that the physical activity must be performed at a particular type of location (e.g., a gymnasium). As another example, the objective may specify that the physical activity must be performed under specified environmental conditions (e.g., within a sufficiently dark room, as determined by determining that a measure of luminosity one or more sensor devices within the environment is less than a defined threshold amount of luminosity). As yet another example, the game application 160 may specify that a particular physical activity (e.g., meditation, where the user must remain sufficiently still for a defined length of time) is to be performed while viewing specified augmented reality animations (e.g., frames depicting villains, ghosts and other scary virtual characters).

The game application 160 determines a pattern of sensor data that constitutes a fitness event (block 615). For example, the game application 160 could determine a pattern of IMU data that represents the user taking a step while carrying the fitness device. As another example, for a fitness activity where the user is tasked with being sufficiently still for a period of time (e.g., meditation), the game application 160 could determine a threshold IMU measurement that, if exceeded, will result in the user failing the fitness objective. Additionally, the game application 160 determines a threshold number of fitness events that must performed for the user to complete the physical world fitness gaming objective (block 620). As an example, the game application 160 could determine that the user must walk 10,000 steps in order to complete the objective. As another example, the game application 160 could determine that the user must perform 50 jumping jack exercises to complete the objective.

In one embodiment, the game application 160 determines a duration for which the user must maintain the pattern of sensor data (e.g., how long the user must perform the physical activity). For instance, the game application 160 could specify that the user must maintain a heartrate above 120 BPM for at least 5 minutes in order to complete the objective. As another example, the game application 160 could determine that the user must maintain an activity level that is less than a threshold level of activity, as indicated by an IMU sensor on the user's person, for at least 5 minutes, in order to complete a particular meditation gaming objective.

The game application 160 then configures one or more fitness devices to monitor the user's progress in completing the objective (block 625). For example, the game application 160 could reset a step counter on a fitness device carried by on the user's person and could configure the fitness device to begin maintaining a tally of when the user's movement, as indicated by one or more IMU sensors and/or accelerometers, matches a pattern of activity corresponding to a user taking a step.

At block 630, the fitness device monitors user activity using one or more sensor devices to collect sensor data. As an example, where the fitness objective is to meditate for a defined period of time, the fitness device could collect data specifying whether the user's movement exceeds a threshold amount of movement and, if so, how long the user was able to maintain a level of movement below the threshold. As another example, the fitness device could collect IMU sensor data as a user walks throughout the physical environment. In the depicted embodiment, the fitness device analyzes the user fitness data to determine occurrences of fitness events, based on the pattern of sensor data (block 635). For example, the fitness device could determine whether the collected IMU sensor data matches a pattern of data characterizing the performance of a step by the user. In such an example, if the collected data matches the pattern, the fitness device could increment a step counter maintained on the fitness device (e.g., within a memory of the fitness device).

At block 640, the fitness device transmits a notification to the gaming application, indicating that a threshold number of fitness events have been performed (block 640). For example, the fitness device could be configured to monitor a threshold number of steps the user has taken and to generate the notification when the maintained step counter on the fitness device exceeds the threshold number of steps. Upon receiving the notification, the game application 160 grants the user one or more in-game rewards (block 645) and the method 600 ends. For example, the gaming application 160 could grant the user's avatar within the computer game a defined reward (e.g., experience points, attribute values, items, abilities, and so on) for successfully completing the fitness objective.

### Technical Description

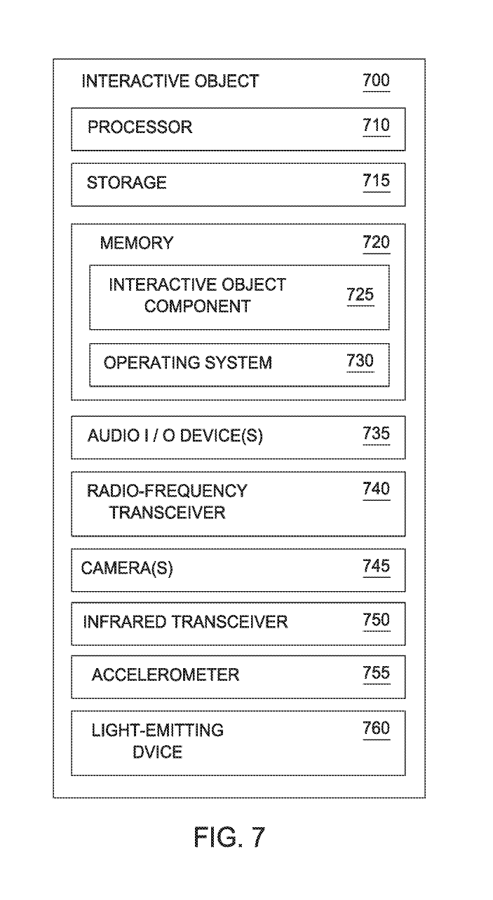

An example of an interactive device is shown in FIG. 7, which is a block diagram illustrating an interactive device configured with an interactive object component, according to one embodiment described herein. In this example, the device 700 includes, without limitation, a processor 710, storage 715, memory 720, audio input/output (I/O) device(s) 735, a radio-frequency (RF) transceiver 740, a camera device(s) 745, an infrared transceiver 750, an accelerometer device 755, and a light-emitting device 760. Generally, the processor 710 retrieves and executes programming instructions stored in the memory 720. Processor 710 is included to be representative of a single CPU, multiple CPUs, a single CPU having multiple processing cores, GPUs having multiple execution paths, and the like. The memory 720 is generally included to be representative of a random access memory. The radio-frequency transceiver 740 enables the interactive object component 725 to connect to a data communications network (e.g., wired Ethernet connection or an 802.11 wireless network). As discussed above, the interactive device may include one or more battery devices (not shown).

Further, while the depicted embodiment illustrates the components of a particular interactive device, one of ordinary skill in the art will recognize that interactive devices may use a variety of different hardware architectures. For instance, in one embodiment the controller component logic is implemented as hardware logic. Examples of such hardware logic include, without limitation, an application-specific integrated circuit (ASIC) and a field-programmable gate array (FPGA). Moreover, it is explicitly contemplated that embodiments may be implemented using any device or computer system capable of performing the functions described herein.

Returning to the embodiment depicted in FIG. 7, the memory 720 represents any memory sufficiently large to hold the necessary programs and data structures. Memory 720 could be one or a combination of memory devices, including Random Access Memory, nonvolatile or backup memory (e.g., programmable or Flash memories, read-only memories, etc.). In addition, memory 720 and storage 715 may be considered to include memory physically located elsewhere; for example, on another computer communicatively coupled to the interactive device 700. Illustratively, the memory 720 includes an interactive object component 725 and an operating system 730. The interactive object component 725 could be configured to receive commands (e.g., encoded in RF or infrared signals) and to execute the commands to perform audiovisual effects. In one embodiment, the interactive object component 725 is configured to decrypt the commands using a received key before executing the commands. The operating system 730 generally controls the execution of application programs on the interactive device 700. Examples of operating system 730 include UNIX, a version of the Microsoft Windows® operating system, and distributions of the Linux® operating system. Additional examples of operating system 730 include custom operating systems for gaming consoles, including the custom operating systems for systems such as the Nintendo DS® and Sony PSP®.

The infrared transceiver 750 represents any device capable of sending and receiving infrared signals. In another embodiment, a device 700 that only sends or receives infrared signals may be configured with an infrared transmitter or a infrared receiver, respectively, as opposed to the infrared transceiver 750. The sound I/O devices 735 could include devices such as microphones and speakers. For example, the speakers could be used to produce sound effects (e.g., explosion sound effects, dialogue, etc.) and/or to produce vibration effects.

Generally, the interactive object component 725 provides logic for the interactive device 700. For example, the interactive object component 725 could be configured to detect that a coded infrared signal has been received (e.g., using the infrared transceiver 750). The interactive object component 725 could then determine a type of the infrared signal (e.g., based on data specified within the coded infrared signal) and could determine a corresponding response based on determined type. For example, the interactive object component 725 could determine that the infrared signal specifies that a ray blast sound effect should be played, and, in response, could output the specified sound effect using audio I/O devices 735. As another example, the signal could be encoded with data specifying that a particular lighting effect should be displayed according to a specified schedule (e.g., at a particular point in time), and the interactive object component 725 could monitor the schedule (e.g., using an internal clock) and could activate the appropriate light-emitting device 760 at the appropriate time.

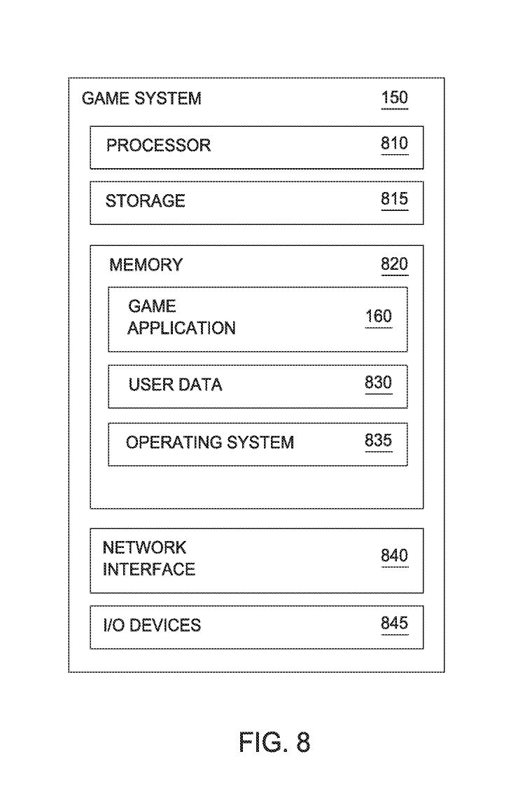

FIG. 8 illustrates an example of a gaming system, according to one embodiment described herein. As shown, the gaming system 150 includes a processor 810, storage 815, memory 820, a network interface 840 and input/output devices 845. Generally, the processor 810 retrieves and executes programming instructions stored in the memory 820. Processor 810 is included to be representative of a single CPU, multiple CPUs, a single CPU having multiple processing cores, GPUs having multiple execution paths, and the like. The memory 820 is generally included to be representative of a random access memory. The network interface 840 enables the gaming system 150 to transmit and receive data across a data communications network. Further, while the depicted embodiment illustrates the components of a particular gaming system 150, one of ordinary skill in the art will recognize that interactive objects may use a variety of different hardware architectures. Moreover, it is explicitly contemplated that embodiments may be implemented using any device or computer system capable of performing the functions described herein.

The memory 820 represents any memory sufficiently large to hold the necessary programs and data structures. Memory 820 could be one or a combination of memory devices, including Random Access Memory, nonvolatile or backup memory (e.g., programmable or Flash memories, read-only memories, etc.). In addition, memory 820 and storage 815 may be considered to include memory physically located elsewhere; for example, on another computer communicatively coupled to the controller device 800. Illustratively, the memory 820 includes a controller component 825, user data 830 and an operating system 835. The operating system 835 generally controls the execution of application programs on the controller device 800. Examples of operating system 835 include UNIX, a version of the Microsoft Windows® operating system, and distributions of the Linux® operating system. Additional examples of operating system 835 include custom operating systems for gaming consoles, including the custom operating systems for systems such as the Nintendo DS® and Sony PSP®.

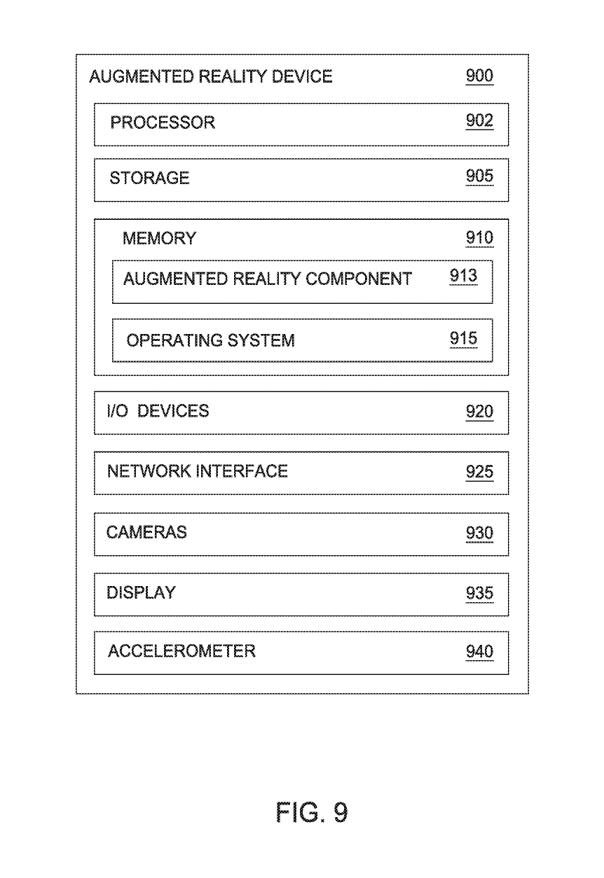

FIG. 9 is a block diagram illustrating a mobile device configured with an augmented reality component, according to one embodiment described herein. In this example, the mobile device 900 includes, without limitation, a processor 902, storage 905, memory 910, I/O devices 920, a network interface 925, camera devices 930, a display devices 935 and an accelerometer device 940. Generally, the processor 902 retrieves and executes programming instructions stored in the memory 910. Processor 902 is included to be representative of a single CPU, multiple CPUs, a single CPU having multiple processing cores, GPUs having multiple execution paths, and the like. The memory 910 is generally included to be representative of a random access memory. The network interface 925 enables the mobile device 900 to connect to a data communications network (e.g., wired Ethernet connection or an 802.11 wireless network). Further, while the depicted embodiment illustrates the components of a particular mobile device 900, one of ordinary skill in the art will recognize that augmented reality devices may use a variety of different hardware architectures. Moreover, it is explicitly contemplated that embodiments of the invention may be implemented using any device or computer system capable of performing the functions described herein.

The memory 910 represents any memory sufficiently large to hold the necessary programs and data structures. Memory 910 could be one or a combination of memory devices, including Random Access Memory, nonvolatile or backup memory (e.g., programmable or Flash memories, read-only memories, etc.). In addition, memory 910 and storage 905 may be considered to include memory physically located elsewhere; for example, on another computer communicatively coupled to the mobile device 900. Illustratively, the memory 910 includes an augmented reality component 913 and an operating system 915. The operating system 915 generally controls the execution of application programs on the augmented reality device 900. Examples of operating system 915 include UNIX, a version of the Microsoft Windows® operating system, and distributions of the Linux® operating system. Additional examples of operating system 915 include custom operating systems for gaming consoles, including the custom operating systems for systems such as the Nintendo DS® and Sony PSP®.

The I/O devices 920 represent a wide variety of input and output devices, including displays, keyboards, touch screens, and so on. For instance, the I/O devices 920 may include a display device used to provide a user interface. As an example, the display may provide a touch sensitive surface allowing the user to select different applications and options within an application (e.g., to select an instance of digital media content to view). Additionally, the I/O devices 920 may include a set of buttons, switches or other physical device mechanisms for controlling the augmented reality device 900. For example, the I/O devices 920 could include a set of directional buttons used to control aspects of a video game played using the augmented reality device 900.

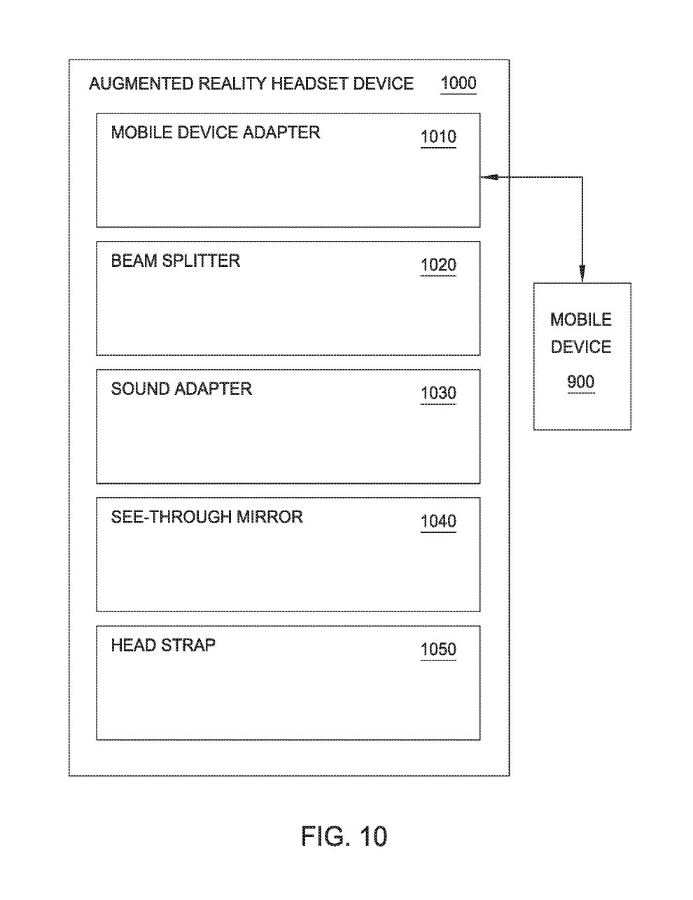

FIG. 10 is a block diagram illustrating an augmented reality headset, according to one embodiment described herein. The augmented reality headset 1000 includes a mobile device adapter 1010, a beam splitter 1020, a sound adapter 1030, a see-through mirror 1040 and a headstrap 1050. Generally, the augmented reality headset device 1000 is configured to interface with a mobile device 900, by way of the mobile device adapter 1010. For example, the mobile device adapter 1010 could be a slot within the augmented reality headset 1000 configured to hold the mobile device 900. The beam splitter 1020 and see-through mirror 1040 are generally arranged in such a way as to project light from the display device 935 of the mobile device 900 to the user's eyes, when the user views the physical environment while wearing the augmented reality headset 1000. For example, the beam splitter 1020 and see-through mirror 1040 could be arranged in the configuration shown in FIG. 3B and discussed above. More generally, however, any configuration suitable for providing an augmented reality display using the light from the display device 935 of the mobile device 900 can be used, consistent with the functionality described herein. The headstrap 1050 generally is used to secure the augmented reality headset 900 to the user's head. More generally, however, any mechanism (e.g., temples that rest atop the user's ears) for securing the augmented reality headset 900 can be used.

In the preceding, reference is made to embodiments of the invention. However, the invention is not limited to specific described embodiments. Instead, any combination of the following features and elements, whether related to different embodiments or not, is contemplated to implement and practice the invention. Furthermore, although embodiments of the invention may achieve advantages over other possible solutions and/or over the prior art, whether or not a particular advantage is achieved by a given embodiment is not limiting of the invention. Thus, the preceding aspects, features, embodiments and advantages are merely illustrative and are not considered elements or limitations of the appended claims except where explicitly recited in a claim(s). Likewise, reference to “the invention” shall not be construed as a generalization of any inventive subject matter disclosed herein and shall not be considered to be an element or limitation of the appended claims except where explicitly recited in a claim(s).

Aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described above with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. Each block of the block diagrams and/or flowchart illustration, and combinations of blocks in the block diagrams and/or flowchart illustration, can be implemented by special-purpose hardware-based systems that perform the specified functions or acts, or combinations of special purpose hardware and computer instructions.

Additional embodiments are described in the attached Appendices A-D, which are hereby incorporated by reference in their entirety. While the foregoing is directed to embodiments of the present invention, other and further embodiments of the invention may be devised without departing from the basic scope thereof, and the scope thereof is determined by the claims that follow.

What is claimed is:

1. A method, comprising:
determining one or more physical world fitness gaming objectives for a first user in a first computer game;
monitoring physical activity of the first user using one or more fitness devices to collect user fitness data;
analyzing the user fitness data collected from the one or more fitness devices to determine whether the first user has completed the one or more physical world gaming objectives; and
upon determining a first one of the one or more physical world gaming objectives has been completed:
determining one or more game rewards corresponding to the completed first physical world gaming objective; and
granting the first user the one or more game rewards within the first computer game.

2. The method of claim 1, wherein analyzing data collected from the one or more fitness devices to determine whether the first user has completed the one or more physical world gaming objectives further comprises:
determining a pattern of sensor data of user fitness data that constitutes an occurrence of a fitness event.

3. The method of claim 2, wherein analyzing data collected from the one or more fitness devices to determine whether the first user has completed the one or more physical world gaming objectives further comprises:
determining a threshold number of fitness events that must be performed to complete the first physical world gaming objective.

4. The method of claim 3, wherein analyzing data collected from the one or more fitness devices to determine whether the first user has completed the one or more physical world gaming objectives further comprises:
analyzing the user fitness data of the first user to determine a number of instances where a respective portion of the user fitness data sufficiently matches the pattern of sensor data.

5. The method of claim 4, wherein determining the first physical world gaming objective have been completed further comprises:
determining that the number of instances where the portion of the user fitness data sufficiently matches the pattern of sensor data is greater than or equal to the threshold number of fitness events that must be performed to complete the first physical world gaming objective.

6. The method of claim 1, wherein determining one or more game rewards corresponding to the completed first physical world gaming objective further comprises:
determining at least one of (i) a measure of experience points, (ii) a virtual item, (iii) a virtual ability, (iv) a virtual follower or pet, (v) an in-game title and (vi) a virtual currency reward, to grant the first user within the first computer game.

7. The method of claim 6, wherein granting the first user the one or more game rewards within the first computer game further comprises:
updating user profile information corresponding to a user account of the first user, to grant the user the one or more game rewards within the first computer game.

8. The method of claim 1, wherein monitoring physical activity of the first user using one or more fitness devices to collect user fitness data further comprises:
determining one or more types of sensor data that the one or more fitness devices are capable of collecting.

9. The method of claim 8, wherein the one or more fitness devices include at least one of one or more accelerometer sensors, one or more inertial measurement sensors, one or more electromyography (EMG) sensors, and one or more heart rate sensors.

10. The method of claim 9, wherein determining one or more types of sensor data that the one or more fitness devices are capable of collecting further comprises determining that the one or more fitness devices are capable of collecting electromyography data, and wherein determining a first one of the one or more physical world gaming objectives have been completed further comprises:
identifying an electromyogram pattern that represents a fitness event for a first one of the one or more physical world gaming objectives within the first computer game;
analyzing the electromyography data collected by the one or more fitness devices to determine a number of fitness events the user has completed; and
upon determining that the number of fitness events exceeds a predefined threshold number of fitness events, determining that the first physical world gaming objective has been completed.

11. The method of claim 1, further comprising:
upon determining the one or more physical world fitness gaming objectives for the first user in the first computer game, providing one or more virtual interactions within the first computer game instructing the first user to perform the one or more physical world fitness gaming objects.

12. The method of claim 11, wherein providing one or more virtual interactions within the first computer game instructing the first user to perform the one or more physical world fitness gaming objects further comprises:
rendering one or more frames depicting a virtual character within an augmented reality environment; and
outputting the one or more frames for display, together with the output of one or more sound effects, by an augmented reality device.

13. The method of claim 1, wherein the one or more physical world gaming objectives comprise performing a physical activity that includes at least one of (i) performing a particular exercise activity a predefined number of times, (ii) walking a predefined number of steps, and (iii) achieving a heartrate that exceeds a predefined threshold level of heartrate.

14. The method of claim 13, wherein the one or more physical world gaming objectives include travelling to a particular physical location, wherein the physical activity must be performed while at the particular physical location in order to complete the one or more physical world gaming objectives.

15. The method of claim 1, wherein the one or more physical world gaming objectives comprise remaining sufficiently still for a period of time.

16. The method of claim 15, wherein the data collected from the one or more fitness devices comprises sensor data indicative of a rate of movement of the one or more fitness devices, wherein analyzing data collected from the one or more fitness devices to determine whether the first user has completed the one or more physical world gaming objectives comprises determining whether sensor data indicates that the rate of movement exceeded a defined threshold rate of movement during the period of time.

17. The method of claim 16, wherein the one or more fitness devices further specify one or more environmental conditions under which the user must remain sufficiently still for the period of time.

18. The method of claim 17, wherein the one or more environmental conditions include at least one of (i) a measure of luminosity within the physical environment, (ii) one or more augmented reality effects being displayed within the physical environment, and (iii) one or more sound effects being output within the physical environment.

19. A non-transitory computer-readable medium containing computer program code that, when executed by operation of one or more computer processors, performs an operation comprising:
determining one or more physical world fitness gaming objectives for a first user in a first computer game;
monitoring physical activity of the first user using one or more fitness devices to collect user fitness data;
analyzing the user fitness data collected from the one or more fitness devices to determine whether the first user has completed the one or more physical world gaming objectives; and
upon determining a first one of the one or more physical world gaming objectives has been completed:
determining one or more game rewards corresponding to the completed first physical world gaming objective; and
granting the first user the one or more game rewards within the first computer game.

20. A system, comprising:
one or more computer processors; and
a non-transitory memory containing computer program code that, when executed by operation of the one or more computer processors, performs an operation comprising:
determining one or more physical world fitness gaming objectives for a first user in a first computer game;
monitoring physical activity of the first user using one or more fitness devices to collect user fitness data;
analyzing the user fitness data collected from the one or more fitness devices to determine whether the first user has completed the one or more physical world gaming objectives; and
upon determining a first one of the one or more physical world gaming objectives has been completed:
determining one or more game rewards corresponding to the completed first physical world gaming objective; and
granting the first user the one or more game rewards within the first computer game.
